(19) United States
(12) Patent Application Publication
(10) Pub. No.: US 2021/0085534 A1
(43) Pub. Date: Mar. 25, 2021

(54) ABSORBENT ARTICLE

(71) Applicant: Kimberly-Clark Worldwide, Inc, Neenah, WI (US)

(72) Inventors: Devin C. McElrath, Broomfield, CO (US); Michael L. Lohoff, Oshkosh, WI (US); Andrew E. Neubauer, Neenah, WI (US); Linda M. Ahles, Neenah, WI (US); Tim G. Dollevoet, Kimberly, WI (US)

(21) Appl. No.: 17/048,176

(22) PCT Filed: Apr. 30, 2019
(86) PCT No.: PCT/US19/30018

§ 371 Date: Oct. 16, 2020

Related U.S. Application Data
(60) Provisional application No. 62/664,711, filed on Apr. 30, 2018.

(51) Int. Cl. A61F 13/514; A61F 13/15; B41M 5/00

(52) U.S. Cl. CPC A61F 13/51496; A61F 13/15577; B41M 5/0064; A61F 2013/8497; B41M 5/007; B41M 5/0076; B41M 5/0058

(57) ABSTRACT

The present disclosure presents a printed absorbent article which includes a hydrophobic substrate, an ink receptive coating and an aqueous ink. The ink receptive coating has a base weight from about 2 gsm to about 20 gsm. The aqueous ink contains pigments. The receptive coating absorbs liquid from the ink at a contact angle at or about zero degrees at which the ink is printed onto the substrate through the receptive coating at a printing speed of at least about 500 feet per minute.

This application claims priority from U.S. provisional Patent Application Ser. No. 62/664711 entitled Container with Ink Receptive Coating, filed on 30 Apr. 2018, the entire contents of which are incorporated herein by reference.

### BACKGROUND OF THE DISCLOSURE

Polymers are used extensively to make a variety of products which include blown and cast films, extruded sheets, injection molded articles, foams, blow molded articles, extruded pipe, monofilaments, fibers, and nonwoven fabrics. Many polymers that are used to form these products, such as polyolefins, are naturally hydrophobic or apolar and are chemically inert. For many uses, hydrophobicity is a disadvantage, particularly when printing with aqueous-based inks having a relatively higher surface tension than the surface energy of the polymeric substrate. For example, aqueous-based inks can have a surface tension of greater than or equal to above a surface tension energy of 45 dynes/cm, while the polymeric substrate may have a surface tension energy of about 30 dynes/cm. Surface tension is the force that holds a fluid together. Surface tension directly impacts whether a coating will wet and spread over, or retract (bead up) from a substrate. While substrate hydrophobicity may not be an issue with lower surface tension inks or solvent-based inks, the apolar nature of the polymeric substrate still will not promote good adhesion of these inks, either aqueous or solvent based, to the polymeric substrates, resulting in printed graphics that will easily rub off when exposed to shear.

Typically, the polymers used to form these products are poorly polar resulting in them being non-conducive to adhere to most common ink compositions that are applied to the surface of the polymeric substrate. Also, these polymers are typically non-absorbent and unable to form a mechanically strong network with the ink composition after it is applied to the polymeric substrate.

Hydrophobic polymers, including polyolefins, such as polyethylene and polypropylene, may be used to manufacture polymeric fabrics which are employed in the construction of packaging articles and disposable absorbent articles such as diapers, feminine care products, incontinence products, training pants, wipes, and so forth. Such polymeric fabrics are often nonwoven fabrics prepared by, for example, processes such as melt blowing, carding, co-forming, spunbonding, and combinations thereof.

Absorbent articles, especially personal care absorbent articles, such as pantiliners, sanitary napkins, interlabial devices, adult incontinent devices, bandages, wipes, diapers, training pants, and swimming pants, typically include an outer cover made from a nonwoven polymeric fabric or alternatively the outer cover is made by printing on a plastic film, then laying a nonwoven on top of the printed plastic film. The outer cover of diapers, training pants, and swimming pants, for example, are difficult to print in a fast and economic manner that is amiable to efficient machine production.

Accordingly, there is a need to improve adhesion of inks to the outer cover substrates of the aforementioned absorbent articles by increasing printing speeds. The current disclosure addresses this need by utilizing a process in which designs and/or graphics may be printed directly onto a small porous size or non-porous nonwoven or alternatively, the designs/graphics are printed onto a plastic film and thereafter a nonwoven is placed on top of the printed film. The novel process disclosed herein may achieve fast printing speeds of at least 500 feet per minute and more without the use of a drying step thus reducing manufacturing process time and cost.

### SUMMARY OF THE DISCLOSURE

The present disclosure presents a printed absorbent article which includes a hydrophobic substrate, an ink receptive coating and an aqueous ink. The ink receptive coating has a base weight from about 2 gsm to about 20 gsm. The aqueous ink contains pigments. The receptive coating absorbs liquid from the ink at a contact angle at or about zero degrees at which the ink is printed onto the substrate at a printing speed of at least about 500 feet per minute.

In accordance with the present disclosure, it has been discovered that the quality of a printed image on an absorbent article may be retained at printing speeds of about 500 feet per minute or more and may reach speeds up to about 2200 feet per minute or more without a separate drying step. By achieving such a fast printing speed without a drying step will reduce manufacturing build out costs and increase efficiency for the user.

In one embodiment of the present disclosure, a printed absorbent article comprises a hydrophobic substrate; an ink receptive coating and an aqueous ink containing pigments. The ink receptive coating has a base weight from about 2 gsm to about 20 gsm. The receptive coating absorbs the liquid, such as water, from the ink at a contact angle at or about zero degrees at which the ink is absorbed onto the substrate. The receptive coating is printed onto the substrate at a printing speed of at least about 500 feet per minute.

It is important to note that the ink receptive coating must have a certain base weight so as to increase absorbency rate, wetting rate of the coating and printing speed. Absorbency and wetting rates of the ink receptive coating as well as printing speeds of the ink and liquid onto the substrate through the ink receptive coating are important measures of the current disclosure. The speed at which the liquid is removed from the ink on the ink receptive coating maybe 10 seconds or less. Or more preferably 5 seconds or less. Or even more preferably 2 seconds or less. Or most preferably 1 second or less. More specifically, during a manufacturing process, the liquefied pigmented ink is applied to the coating whereby the liquid, such as water, is absorbed by the coating and the ink may then be absorbed onto the substrate thus eliminating the use of a drying apparatus.

In another embodiment, an ink receptive coating may have an original base weight of about 6 gsm whereby the ink is printed onto the substrate though the coating at a printing speed of at least about 800 feet per minute.

In still a further embodiment, an ink receptive coating may have an original base weight of about 8 gsm whereby the ink is printed onto the substrate though the coating at a printing speed of at least about 900 feet per minute.

In another embodiment, an ink receptive coating may have an original base weight of about 10 gsm whereby the ink is printed onto the substrate though the coating at a printing speed of at least about 1,000 feet per minute.

In yet a further embodiment, an ink receptive coating may have an original base weight of about 12 gsm whereby the ink is printed onto the substrate though the coating at a printing speed of at least about 1,000 feet per minute.

In still another embodiment, an ink receptive coating may have an original base weight of about 14 gsm whereby the ink is printed onto the substrate though the coating at a printing speed of at least about 1,200 feet per minute.

In yet another embodiment, an ink receptive coating may have an original base weight of about 16 gsm whereby the ink is printed onto the substrate though the coating at a printing speed of at least about 1,400 feet per minute.

In a further embodiment, an ink receptive coating may have an original base weight of about 18 gsm whereby the ink is printed onto the substrate though the coating at a printing speed of at least about 1,700 feet per minute.

And yet another embodiment, an ink receptive coating may have an original base weight of about 20 gsm whereby the ink is printed onto the substrate though the coating at a printing speed of at least about 2,000 feet per minute.

In an additional embodiment, the printed absorbent article, according to the preceding embodiments, wherein a design or graphic being printed onto the absorbent article is done by sparse printing on an absorbent article of a printed package. By printing on such a surface will provide a smoother image/graphic and/or design on the final printed absorbent article.

In yet another embodiment of the present disclosure, the process of making a printed absorbent article may comprise applying an ink receptive coating to a hydrophobic substrate. The ink receptive coating has a base weight from about 2 gsm to about 20 gsm. An aqueous ink containing pigments is applied to a substrate at a printing speed of at least about 500 feet per minute. The ink receptive coating absorbs liquid from the ink at a contact angle at or about zero degrees.

In an additional embodiment, according to the preceding embodiment, wherein the printing speed is about or greater than 1000 feet per minute, is about or greater than 1400 feet per minute, is about or greater than 2000 feet per minute.

### BRIEF DESCRIPTION OF DRAWINGS

The disclosure may be more completely understood in consideration of the following detailed description of various embodiments in connection with the accompanying drawing, in which:

FIG. 1 shows a comparison between two disposable absorbent articles. The article on the left side depicts wet ink transferred to the backside of the printed surface and the article on the right side depicts the absence of any ink transferring to the backside of the printed surface.

FIG. 2 illustrates an infant disposable diaper.

### DETAILED DESCRIPTION OF THE DISLOSURE

When introducing elements of the present disclosure or the preferred embodiment(s) thereof, the articles “a”, “an”, and “the” are intended to mean that there are one or more of the elements.

The terms “comprising”, “including” and “having” are intended to be inclusive and mean that there may be additional elements other than the listed elements.

The term “absorbent article” refers to devices that absorb and contain body exudates, and, more specifically, refers to devices that are placed against or in proximity to the body of the wearer to absorb and contain the various exudates discharged from the body. Absorbent articles may include diapers, training pants, adult incontinence undergarments, feminine hygiene products, breast pads, care mats, bibs, wound dressing products, and the like. As used herein, the term “body exudates” includes, but is not limited to, urine, blood, vaginal discharges, breast milk, sweat and fecal matter.

The term “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. Nonwovens may include hydroentangled nonwovens. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes such as meltblowing, spunbonding, solvent spinning, electrospinning, and carding. The basis weight of nonwoven fabrics is usually expressed in grams per square meter (gsm).

The term “substrate” includes any material that the inks of the present invention can be printed on. Thus, substrates of the present invention include, but are not limited to, nonwovens, films, fibrous polyolefin webs, polyolefin webs, cellulosic webs, elastomeric webs, laminates of one or more of the above or any combination of one or more of the above.

The term “sparse printing” is defined as a low base weight of ink wherein porosity is small enough to limit the spreading of ink and/or liquid on an ink receptive coating such as a plastic film or tiny or non-porous non-woven.

### Absorbent Article

For example purposes a diaper is disclosed herein. The use of applying the hydrophobic substrate, ink receptive coating and aqueous ink disclosed herein may be applied to an outer cover of a diaper made from a nonwoven polymeric fabric. Or alternatively, applying the hydrophobic substrate, ink receptive coating and aqueous ink to the outer cover of the diaper is made by printing on a plastic film, then laying the nonwoven fabric on top of the printed plastic film. Absorbent articles, other than a diaper, where applying the hydrophobic substrate, ink receptive coating and aqueous ink to the outer cover or plastic film as described above include pantiliners, sanitary napkins, interlabial devices, adult incontinent devices, bandages, wipes, training pants, and swimming pants.

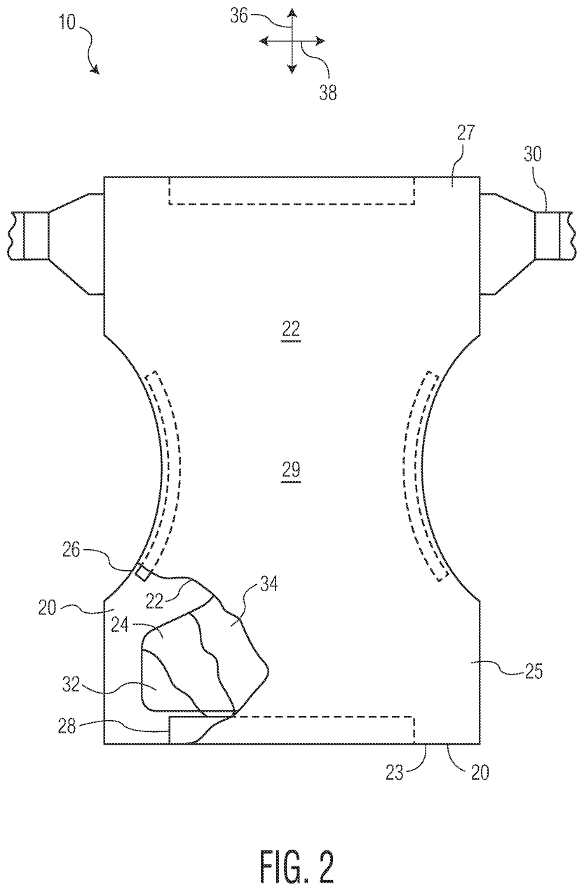

A typical absorbent article will be explained with reference to FIG. 2. FIG. 2 illustrates an exemplary disposable absorbent article 10 that is an infant disposable diaper. The example of the use of the disposable diaper for infants is intended to be representative and not limiting. The disposable absorbent article 10 includes a backsheet or (outer cover) 20, a liquid permeable topsheet (or bodyside liner) 22 positioned in facing relation with the backsheet 20, and an absorbent core 24, such as an absorbent pad, that is located between the topsheet 22 and the backsheet 20. The article 10 has an outer surface 23, a front waist region 25, a back waist region 27, and a crotch region 29 connecting the front and back waist regions 25, 27. The backsheet 20 defines a length and a width that, in the illustrated aspect, coincide with the length and width of the article 10. The absorbent core 24 generally defines a length and width that are less than the length and width of the backsheet 20, respectively. Thus, marginal portions of the article 10, such as marginal sections of the backsheet 20, can extend past the terminal edges of the absorbent core 24. In the illustrated aspects, for example, the backsheet 20 extends outwardly beyond the terminal marginal edges of the absorbent core 24 to form side margins and end margins of the article 10. The topsheet 22 is generally coextensive with the backsheet 20 but can optionally cover an area that is larger or smaller than the area of the backsheet 20, as desired. In other words, the topsheet 22 is connected in superposed relation to the backsheet 20. The backsheet 20 and topsheet 22 are intended to face the garment and body of the wearer, respectively, while in use.

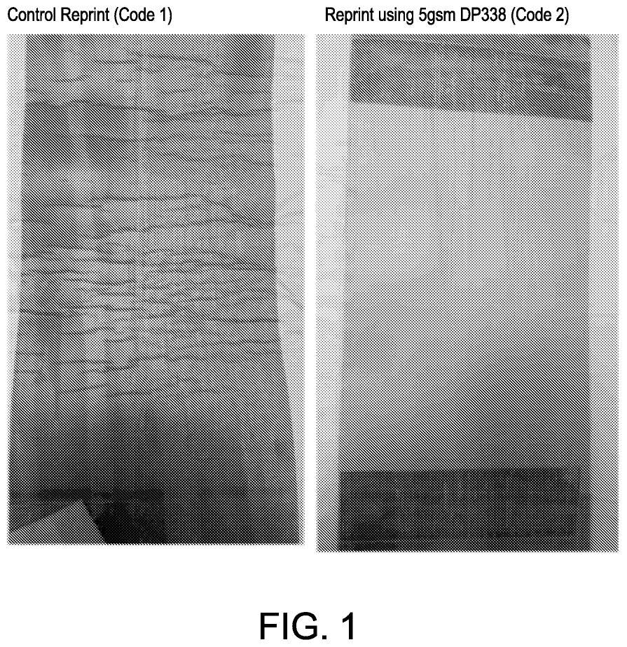

To provide improved fit and to help reduce leakage of body exudates from the article 10, the article side margins and end margins can be elasticized with suitable elastic members, such as single or multiple strands of elastic. The elastic strands can be composed of natural or synthetic rubber and can optionally be heat shrinkable or heat elasticizable. For example, as representatively illustrated in FIG. 1, the article 10 can include leg elastics 26 that are constructed to operably gather and shirr the side margins of the article 10 to provide elasticized leg bands that can closely fit around the legs of the wearer to reduce leakage and provide improved comfort and appearance. Similarly, waist elastics 28 can be employed to elasticize the end margins of the article 10 to provide elasticized waists. The waist elastics 28 are configured to operably gather and shirr the waist sections to provide a resilient comfortably close fit around the waist of the wearer. In the illustrated aspects, the elastic members are illustrated in their uncontracted, stretched condition for the purpose of clarity.

Fastening means, such as hook and loop fasteners 30, may be employed to secure the article 10 on a wearer. Alternatively, other fastening means, such as buttons, pins, snaps, adhesive tape fasteners, cohesives, mushroom-and-loop fasteners, a belt, and so forth, as well as combinations including at least one of the foregoing fasteners can be employed. Additionally, more than two fasteners can be provided, particularly if the article 10 is to be provided in a prefastened configuration.

The article 10 may further include other layers between the absorbent core 24 and the topsheet 22 or backsheet 20. For example, article 10 may also include a surge management layer 34 located between the topsheet 22 and the absorbent core 24 to prevent pooling of the fluid exudates and further improve air exchange and distribution of the fluid exudates within the article 10.

The article 10 may be of various suitable shapes. For example, the article 10 may have an overall rectangular shape, T-shape or an approximately hourglass shape. In the shown aspect, the article 10 has a generally I-shape. The article 10 further defines a longitudinal direction 36 and a transverse direction 38. Other suitable article components that can be incorporated on absorbent articles include containment flaps, waist flaps, elastomeric side panels, and the like. Examples of possible article configurations are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,192,606 issued Mar. 9, 1993, to Proxmire et al., and U.S. Pat. No. 5,509,915 issued Apr. 23, 1996 to Hanson et al.

The various components of the article 10 are integrally assembled employing various types of attachment mechanisms such as adhesive, sonic bonds, thermal bonds, and so forth, as well as combinations including at least one of foregoing mechanisms. In the shown aspect, for example, the topsheet 22 and backsheet 20 are assembled to the absorbent core 24 with lines of adhesive, such as a hot melt, pressure-sensitive adhesive. Similarly, other article components, such as the elastic members 26 and 28, fastening members 30, and surge layers 34 can be assembled into the article 10 by employing the above-identified attachment mechanisms.

The backsheet 20 of the article 10 may include any material used for such applications, such as a substantially vapor-permeable material. The permeability of the backsheet 20 may be configured to enhance the breathability of the article 10 and to reduce the hydration of the wearer's skin during use without allowing excessive condensation of vapor, such as urine, on the garment facing surface of the backsheet 20 that can undesirably dampen the wearer's clothes. The backsheet 20 can be constructed to be permeable to at least water vapor and can have a water vapor transmission rate of greater than or equal to about 1,000 grams per square meter per 24 hours (g/m²/24 hr). For example, the backsheet 20 can define a water vapor transmission rate of about 1,000 to about 6,000 g/m²/24 hr.

The backsheet 20 is also desirably substantially liquid impermeable. For example, the backsheet 20 can be constructed to provide a hydrohead value of greater than or equal to about 60 centimeters (cm), or, more specifically, greater than or equal to about 80 cm, and even more specifically, greater than or equal to about 100 cm. A suitable technique for determining the resistance of a material to liquid penetration is Federal Test Method Standard (FTMS) 191 Method 5514, dated Dec. 31, 1968.

As stated above, the backsheet 20 may include any material used for such applications, and desirably includes materials that either directly provide the above desired levels of liquid impermeability and air permeability and/or materials that can be modified or treated in some manner to provide such levels. The backsheet 20 can be a nonwoven fibrous web constructed to provide the required level of liquid impermeability. For example, a nonwoven web including spunbond and/or meltblown polymer fibers can be selectively treated with a water repellent coating and/or laminated with a liquid impermeable, vapor permeable polymer film to provide the backsheet 20. In another aspect, the backsheet 20 can include a nonwoven web including a plurality of randomly deposited hydrophobic thermoplastic meltblown fibers that are sufficiently bonded or otherwise connected to one another to provide a substantially vapor permeable and substantially liquid impermeable web. The backsheet 20 can also include a vapor permeable nonwoven layer that has been partially coated or otherwise configured to provide liquid impermeability in selected areas. In yet another example, the backsheet 20 is provided by an extensible material. Further, the backsheet 20 material can have stretch in the longitudinal 36 and/or transverse 38 directions. When the backsheet 20 is made from extensible or stretchable materials, the article 10 provides additional benefits to the wearer including improved fit.

The topsheet 22, employed to help isolate the wearer's skin from liquids held in the absorbent core 24, can define a compliant, soft, non-irritating feel to the wearer's skin. Further, the topsheet 22 can be less hydrophilic than the absorbent core 24, to present a relatively dry surface to the wearer, and can be sufficiently porous to be liquid permeable, permitting liquid to readily penetrate through its thickness. A suitable topsheet 22 may be manufactured from a wide selection of web materials, such as porous foams, reticulated foams, apertured plastic films, natural fibers (for example, wood or cotton fibers), synthetic fibers (for example, polyester or polypropylene fibers), and the like, as well as a combination of materials including at least one of the foregoing materials.

Various woven and nonwoven fabrics may be used for the topsheet 22. For example, the topsheet 22 may include a meltblown or spunbond web (e.g., of polyolefin fibers), a bonded-carded web (e.g., of natural and/or synthetic fibers), a substantially hydrophobic material (e.g., treated with a surfactant or otherwise processed to impart a desired level of wettability and hydrophilicity), and the like, as well as combinations including at least one of the foregoing. For example, the topsheet 22 can include a nonwoven, spunbond, polypropylene fabric, optionally including about 2.8 to about 3.2 denier fibers formed into a web having a basis weight of about 22 grams per square meter (g/m²) and a density of about 0.06 gram per cubic centimeter (g/cc).

The absorbent core 24 of the article 10 may include a matrix of hydrophilic fibers, such as a fibrous web of cellulosic fibers. The wood pulp fluff can be exchanged with synthetic, polymeric, meltblown fibers, and the like, as well as a combination including at least one of the foregoing. Alternatively, the absorbent core 24 can include a laminate of fibrous webs and/or a suitable matrix. When the absorbent core 24 includes hydrophilic fibers they can form an average basis weight for the absorbent core 24 that may be about 300 grams per square meter (g/m²) to about 900 g/m², or, more specifically, about 500 g/m² to about 800 g/m², and even more specifically, about 550 g/m² to about 750 g/m².

Optionally, the absorbent core 24 may further include a support (e.g., a substantially hydrophilic tissue or nonwoven wrap sheet (not illustrated)) to help maintain the integrity of the structure of the absorbent core 24. The tissue wrapsheet may be placed about the web/sheet of high-absorbency material and/or fibers, optionally over at least one or both major facing surfaces thereof. The tissue wrapsheet can include an absorbent cellulosic material, such as creped wadding or a high wet-strength tissue. The tissue wrapsheet may optionally be configured to provide a wicking layer that helps to rapidly distribute liquid over the mass of absorbent fibers constituting the absorbent core 24. If this support is employed, the colorant 40 may optionally be disposed in the support, on the side of the absorbent core 24 opposite the backsheet 20.

Due to the thinness of absorbent core 24 and the high absorbency material within the absorbent core 24, the liquid uptake rates of the absorbent core 24, by itself, can be too low, or cannot be adequately sustained over multiple insults of liquid into the absorbent core 24. To improve the overall liquid uptake and air exchange, the article 10 can further include a porous, liquid-permeable layer or surge management layer 34, as representatively illustrated in FIG. 2. The surge management layer 34 is typically less hydrophilic than the absorbent core 24, and can have an operable level of density and basis weight to quickly collect and temporarily hold liquid surges, to transport the liquid from its initial entrance point and to substantially completely release the liquid to other parts of the absorbent core 24. This configuration can help prevent the liquid from pooling and collecting on the portion of the article 10 positioned against the wearer's skin, thereby reducing the feeling of wetness by the wearer. The structure of the surge management layer 34 can also enhance the air exchange within the article 10.

Various woven and nonwoven fabrics may be used to construct the surge management layer 34. For example, the surge management layer 34 can be a layer including a meltblown or spunbond web of synthetic fibers (such as polyolefin fibers); a bonded-carded-web or an airlaid web including, for example, natural and/or synthetic fibers; hydrophobic material that is optionally treated with a surfactant or otherwise processed to impart a desired level of wettability and hydrophilicity; and the like, as well as combinations including at least one of the foregoing. The bonded carded-web can, for example, be a thermally bonded web that is bonded using low melt binder fibers, powder, and/or adhesive. The layer can optionally include a mixture of different fibers. For example, the surge management layer 34 can include a hydrophobic, nonwoven material having a basis weight of about 30 to about 120 g/m².

The backsheet 20 desirably comprises a material that is substantially liquid impermeable, and may be elastic, stretchable or nonstretchable. The backsheet 20 may be a single layer of liquid impermeable material, but desirably comprises a multi-layered laminate structure in which at least one of the layers is liquid impermeable. For instance, the backsheet 20 may include a liquid permeable outer layer and a liquid impermeable inner layer that are suitably joined together by a laminate adhesive (not shown). Suitable laminate adhesives, which may be applied continuously or intermittently as beads, a spray, parallel swirls, or the like, can be obtained from Findley Adhesives, Inc., of Wauwatosa, Wis., U.S.A., or from National Starch and Chemical Company, Bridgewater, N.J., U.S.A. The liquid permeable outer layer can be any suitable material and desirably one that provides a generally cloth-like texture. One example of such a material is a 20 gsm (grams per square meter) spunbond polypropylene nonwoven web. The outer layer may also be made of those materials of which liquid permeable topsheet 22 is made. While it is not a necessity for outer layer to be liquid permeable, it is desired that it provides a relatively cloth-like texture to the wearer.

The inner layer of the backsheet 20 may be both liquid and vapor impermeable, or may be liquid impermeable and vapor permeable. The inner layer is desirably manufactured from a thin plastic film, although other flexible liquid impermeable materials may also be used. The inner layer, or the liquid impermeable backsheet 20 when a single layer, prevents waste material from wetting articles, such as bedsheets and clothing, as well as the wearer and caregiver. A suitable liquid impermeable film for use as a liquid impermeable inner layer, or a single layer liquid impermeable backsheet 20, is a 1.0 mil polyethylene film commercially available from Edison Plastics Company of South Plainfield, N.J., U.S.A. If the backsheet 20 is a single layer of material, it can be embossed and/or matte finished to provide a more cloth-like appearance. As earlier mentioned, the liquid impermeable material can permit vapors to escape from the interior of the disposable absorbent article, while still preventing liquids from passing through the backsheet 20. A suitable “breathable” material is composed of a microporous polymer film or a nonwoven fabric that has been coated or otherwise treated to impart a desired level of liquid impermeability. A suitable microporous film is a PMP-1 film material commercially available from Mitsui Toatsu Chemicals, Inc., Tokyo, Japan, or an XKO-8044 polyolefin film commercially available from 3M Company, Minneapolis, Minn., U.S.A.

The liquid permeable topsheet 22 is illustrated as overlying the backsheet 20 and may but need not have the same dimensions as the backsheet 20. The topsheet 22 is desirably compliant, soft feeling, and non-irritating to the child's skin.

The topsheet 22 may be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. Various woven and nonwoven fabrics may be used for the topsheet 22. For example, the topsheet may be composed of a meltblown or spunbonded web of polyolefin fibers. The topsheet may also be a bonded-carded web composed of natural and/or synthetic fibers.

The topsheet 22 may be composed of a substantially hydrophobic material, and the hydrophobic material may, optionally, be treated with a surfactant or otherwise processed to impart a desired level of wettability and hydrophilicity. For example, the material may be surface treated with about 0.28 weight percent of a surfactant commercially available from the Rohm and Haas Co. under the trade designation Triton X-102. The surfactant may be applied by any conventional means, such as spraying, printing, brush coating or the like. The surfactant may be applied to the entire topsheet 22 or can be selectively applied to particular sections of the topsheet 22, such as the medial section along the longitudinal centerline.

Alternatively, a suitable liquid permeable topsheet 22 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber available from CHISSO Corporation, Osaka, Japan. In this particular bicomponent fiber, the polypropylene forms the core and the polyethylene forms the sheath of the fiber. Other fiber orientations are possible, such as multi-lobe, side-by-side, end-to-end, or the like. While the backsheet 20 and topsheet 22 may comprise elastomeric materials, it can be desirable in some embodiments for the composite structure to be generally inelastic, where the top sheet, the backsheet 20 and the absorbent core 24 comprise materials that are generally not elastomeric.

Suitable elastic materials are described in the following U.S. patents: U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. In particular embodiments, the elastic material comprises a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Mormon; and European Patent Application No. EP 0 217 032 published on Apr. 8, 1987 in the names of Taylor et al.; all of which are incorporated herein by reference.

The absorbent core 24 may include suitable superabsorbent polymers (or materials) capable of absorbing moisture may be selected from natural, synthetic, and modified natural polymers and materials. The superabsorbent materials can be inorganic materials, such as silica gels, or organic compounds, such as crosslinked polymers.

Absorbing Pigment onto a Substrate Without Using Drying Ovens

Industry standards pertaining to aqueous ink technologies require that pigments absorb onto a substrate while water is removed by evaporation in drying ovens. To adequately dry these aqueous inks at process speeds at or below 2000 feet per minute, the drying ovens would have to be at least 40 feet or more. Most manufacturing lines do not have enough space to accommodate these types of large drying ovens nor is it economically feasible. And, moreover, even if these manufacturing lines could be modified to accommodate these large drying ovens, the cost of their operation and the potential impact on print quality would be risky and too costly to justify the expense needed to implement such a manufacturing line. Therefore, a solution is needed that does not require these large drying ovens.

Surprisingly and unexpectedly, the current disclosure has found a solution to the aforementioned without using drying ovens. The solution for absorbing the pigment onto a substrate is not to evaporate a liquid with large drying ovens, but to spread and absorb the liquid, such as water and pigment(s), with an absorbent ink receptive coating onto the substrate.

Substrates disclosed herein are preferably very hydrophobic such as untreated polyethylene and polypropylene. Substrates may also be treated with other ingredients to improve the adherence of the coating to the substrate. Corona treating the substrate to increase the adherence of the coating to the substrate is used herein as well to improve the adherence of the coating to the substrate. Corona treatment must be done at an optimal time. If corona treatment is done too long it will destroy the properties of the coating and surface of the substrate. Other suitable printing processes may be used such as digital ink jet printing for the disclosure herein.

### Ink Receptive Coating

The ink receptive coating needs to be thick enough to absorb the liquid, such as water, contained in the aqueous ink. The ink receptive coating has a base weight from about 2 gsm to about 20 gsm. The base weight is reduced on average from about 25% to about 38% from its original weight after a liquid is absorbed into the ink receptive coating.

Suitable ink receptive coatings include but not limited to are absorbent materials like talc, cellulose fibers, superabsorbent for example to aid in absorption. The coating may also contain a wetting agent like a surfactant to ensure a near zero contact angle when the inks come in contact with the coating. The coating may include other and the coating to the substrate to improve both dry and wet crockfastness.

### Aqueous Ink Composition

Embodiments of the present invention includes an ink composition comprising a water-based ink. The ink composition may include a water-based polymer, a binder component, resolubility agent, pigments and optionally wax and/or lubricants. Examples of water-based inks useful in the present disclosure are available from Environmental Inks and Coatings Corporation, Morganton, N.C., under the following code numbers: EH034677 (yellow); EH057960 (magenta); EH028676 (cyan); EH092391 (black); EH034676 (orange); and EH064447 (green) and any similar inks thereof. Other suitable coatings are inkjet coatings such as NuCoat Digitall 9191 MIJ or Lubrizol DP338.

The aqueous ink composition may include polymers such as acrylics, acrylic latex, styrenated acrylics, ethylene vinyl acetate, ethylene vinyl chlorides and styrene butadiene rubbers (SBR's) or any combinations thereof.

The aqueous ink composition may include a binder component. The binder component may be from about 20 to about 45 weight percent of the ink composition. In one embodiment of the present invention, the binder component may be a polyurethane dispersion (also known herein as “PUD”). The PUD may be a high elongation, high tensile strength, high hardness, water-based polymeric dispersion.

The aqueous ink may include a wax component. The wax component may be from about 8 to about 18 weight percent of the aqueous ink composition. A wax or wax blend may be useful in the present invention. Appropriate waxes/blends include polyethylene, carnauba, paraffin, silicone oil, polypropylene, polyolefin blends and combinations thereof.

The aqueous ink may include a resolubility agent. The resolubility agent may be from about 5.0 to about 16.0 weight percent of the ink composition. Resolubility agents useful in the present invention include acrylics solutions and dispersions with a high to medium degree of carboxyl functionality. In one embodiment, medium acid number, acrylic colloidal dispersion resolubility agents are useful in the present disclosure.

The aqueous ink composition may also include additional waxes and lubricants for detackification and lowering CoF. The additional wax/lubricant blend may be comprised of carnauba (the wax) and silicone oil (the lubricant). In one embodiment, the wax/lubricant blend is from about 1 to about 4 weight percent of the composition. Waxes useful in the present invention include polyethylene, polypropylenes, high density polyethylene, low density polyethylene and paraffin.

### Pigmented Aqueous Ink Compositions

The aqueous ink may include pigments. Examples of suitable pigments include, but are not limited to, Blue 15:3, Violet 23, Violet 27, Yellow 14, Yellow 74, Yellow, 83, Yellow 97, Yellow 13, Green 7, Red 2, Red 22, Red 48:1, Red 57:1, Red 122, Red 184, Red 238, Red 269, Red 49:1, Red 81:1 Red 49:2, Red 166, Red 170, Orange 5, Orange 16, Orange 46, White 7, Black 7, iron oxides, and combinations thereof. In one embodiment, from about 10 to about 16 weight percent pigments are employed, but this may vary according to the specific color and desired density. In one embodiment, pigments in a colloidal dispersion, collectively a colorant, are useful in the present disclosure.

The printing of substrates, such as woven and nonwoven fabrics and films, is well known. The printing of fabrics with inks and dyes is a common and widely used method for imparting patterns and colors to a basic fabric. Many current products, such as diapers and training pants, include printed graphics to improve their appearance. A problem with such printed products is that the printed graphics may be smeared or even removed during the handling of products during manufacturing, packaging, and use.

Pigmented inks are beneficial for use on substrates because they tend to be more resistant to leaching and mechanical rubbing than dye-based inks and thus tend to be more resistant to being removed from the surface of substrates. Pigment-based inks also have better optical density per unit weight (better “mileage”) than dye-based inks, meaning that less pigment-based ink is required to create the intensity of color. However, adhesive components are necessary to use along with pigmented inks to prevent removal of the pigment from the surface by mechanical abrasion or chemical leaching.

The industrial importance of pigment-based inks has increased in recent times. This is driven, in part, by the development of many new synthetic substrates that are incapable of being printed with conventional solvent-based or water-based inks, and consumer preferences that their goods be printed with brand identifiers, aesthetically pleasing designs or functional markings. In order to adapt pigment-based inks for use in a variety of applications, namely low surface tension substrates, others have employed high loads of volatile organic compounds (“VOC's”), thereby reducing the static and dynamic surface tension of the inks. However, volatile organic compounds such as alcohols, esters, ketones, aromatics and aliphatics create environmental hazards in their production, disposal and use. They are also expensive. One example of an ink used on a low surface tension substrate is set forth in U.S. Pat. No. 5,458,590 to Schleinz et al., which employs a solvent blend to impart the desired surface tension to the ink.

The aqueous ink composition may include surfactants. Surfactants may be present in the range of from about 1.0 to about 10.0 weight percent of the ink composition. Surfactants useful in the present invention include dioctyl sulfosuccinates, phosphate esters, alkoxylated alcohols, ethoxylated diols, and mixtures or blends thereof.

The aqueous ink composition may be applied to the substrate by any method known in the art. Specifically, the ink composition may be applied to the substrate using ink jet printers, flexographic printing presses, gravure printing presses, or a combination thereof. The aqueous ink composition may be printed on a number of article components including, but not limited to, the backsheet, topsheet, cuffs, etc. In one embodiment, the aqueous ink composition is applied to the substrate by flexographic or rotogravure printing. A metering roll or doctor blade system may be used.

### EXAMPLE

One example of preparing an ink absorbent article as disclosed herein is to add an absorbent layer onto a polyolefin film. One way to achieve this is by taping talc powder, tissue (cellulose fibers), and absorbent fabrics onto a polyolefin film. For comparison, the industry standard is to print on corona or plasma treated polyolefin films. FIG. 1 shows the results of taping talc powder, tissue, and absorbent fabrics onto a polyolefin film. Specifically, FIG. 1 shows a comparison between two disposable absorbent articles. The left side absorbent article shows the presence of a wet ink transferred to the backside of the printed surface. The right side absorbent article shows the absence of any ink transferring to the backside of the printed surface.

The utilization NuCoat Digitall 9191 as a print receptive coating has been shown to increase the print speed without the use of a dryer. Specifically it was found that using the Nu Coat material at a base weight of 12 gsm would yield about 1400 fpm printing speed. This result was gathered without the use of a corona treater.

### EMBODIMENTS

1. A printed absorbent article comprising:
hydrophobic substrate;
ink receptive coating wherein the coating has a base weight from about 2 gsm to about 20 gsm;
aqueous ink containing pigments wherein the coating absorbs liquid from the ink at a contact angle at or about zero degrees and the ink is absorbed and printed onto the substrate at a printing speed of at least about 500 feet per minute.

2. The printed absorbent article according to claim 1, wherein the substrate is a polyolefin or a plastic film.

3. The printed absorbent article of claims 1-2, wherein the substrate comprises paper, wood, woven fabric, textile, plastic, glass, metal, foil, or a combination thereof.

4. The printed absorbent article according to claims 1-3, wherein the ink receptive coating comprises talc, cellulose fibers, superabsorbent amorphous silica or a combination thereof.

5. The printed absorbent article according to claims 1-4, wherein the ink receptive coating comprises a surfactant.

6. The printed absorbent article according to claims 1-5, wherein the printing speed is about 1400 feet per minute and up to about 2200 feet per minute.

7. The printed absorbent article according to claims 1-6, wherein the amount of aqueous ink applied to the substrate through the coating is dependent on the amount of ink required to prepare a graphic, design or number onto the substrate.

8. The printed absorbent article according to claims 1-7, wherein the amount of aqueous ink absorbed by the coating after applying the aqueous ink onto the substrate is the same.

9. The printed absorbent article according to claims 1-8, wherein the printed absorbent article is packaged for commercial sale.

10. The printed absorbent article according to claims 1-9, wherein the printed absorbent article is a pantiliners, sanitary napkins, interlabial devices, adult incontinence devices, bandages, wipes, diapers and the like.

11. A printing process for making a printed absorbent article comprising the steps:
applying an ink receptive coating to a substrate wherein the coating has a base weight from about 2 gsm to about 20 gsm:
applying aqueous ink containing pigments, whereby the receptive coating absorbs liquid from the ink at a contact angle at or about zero degrees, onto the substrate at a printing speed of at least about 500 feet per minute.

12. The printing process for making a printed absorbent article according to claim 10, wherein the substrate is a polyolefin or a plastic film.

13. The printing process for making a printed absorbent article according to claims 11-12, wherein the substrate comprises paper, wood, woven fabric, textile, plastic, glass, metal, foil, or a combination thereof.

14. The printing process for making a printed absorbent article according to claims 11-13, wherein the ink receptive coating comprises talc, cellulose fibers, superabsorbent amorphous silica or a combination thereof.

15. The printing process for making a printed absorbent article according to claims 11-14, wherein the ink receptive coating comprises a surfactant.

16. The printing process for making a printed absorbent article according to claims 11-15, wherein the printing speed may be about 1000, about 1400, or about 2200 feet per minute.

17. The printing process for making a printed absorbent article according to claims 11-16, wherein the amount of aqueous ink applied to the substrate through the coating is dependent on the amount of ink required to prepare a graphic, design or number onto the substrate.

18. The printing process for making a printed absorbent article according to claims 11-17, wherein the amount of aqueous ink absorbed by the coating after applying the aqueous ink onto the substrate is the same.

19. The printing process for making a printed absorbent article according to claims 11-18, wherein the printed absorbent article is packaged for commercial sale.

1. A printed absorbent article comprising:
hydrophobic substrate;
ink receptive coating wherein the coating has a base weight from about 2 gsm to about 20 gsm;
aqueous ink containing pigments wherein the ink receptive coating absorbs liquid from the ink at a contact angle at or about zero degrees and the ink is absorbed and printed onto the hydrophobic substrate at a printing speed of at least about 500 feet per minute.

2. The printed absorbent article according to claim 1, wherein the hydrophobic substrate is a polyolefin or a plastic film.

3. The printed absorbent article of claim 1, wherein the hydrophobic substrate comprises paper, wood, woven fabric, textile, plastic, glass, metal, foil, or a combination thereof.

4. The printed absorbent article according to claim 1, wherein the ink receptive coating comprises talc, cellulose fibers, superabsorbent amorphous silica or a combination thereof.

5. The printed absorbent article according to claim 1, wherein the ink receptive coating comprises a surfactant.

6. The printed absorbent article according to claim 1, wherein the printing speed is from about 1400 feet per minute to about 2200 feet per minute.

7. The printed absorbent article according to claim 1, wherein the amount of aqueous ink applied to the hydrophobic substrate through the ink receptive coating is dependent on the amount of ink required to prepare a graphic, design or number onto the substrate.

8. The printed absorbent article according to claim 1, wherein the amount of aqueous ink absorbed by the ink receptive coating after applying the aqueous ink onto the hydrophobic substrate is the same.

9. The printed absorbent article according to claim 1, wherein the printed absorbent article is packaged for commercial sale.

10. The printed absorbent article according to claim 1, wherein the printed absorbent article is a pantiliners, sanitary napkins, interlabial devices, adult incontinence devices, bandages, wipes, diapers and the like.

11. A printing process for making a printed absorbent article comprising the steps:
applying an ink receptive coating to a substrate wherein the coating has a base weight from about 2 gsm to about 20 gsm:
applying aqueous ink containing pigments, whereby the receptive coating absorbs liquid from the ink at a contact angle at or about zero degrees, onto the substrate at a printing speed of at least about 500 feet per minute.

12. The printing process for making a printed absorbent article according to claim 11, wherein the substrate is a polyolefin or a plastic film.

13. The printing process for making a printed absorbent article according to claim 11, wherein the substrate comprises paper, wood, woven fabric, textile, plastic, glass, metal, foil, or a combination thereof.

14. The printing process for making a printed absorbent article according to claim 11, wherein the ink receptive coating comprises talc, cellulose fibers, superabsorbent amorphous silica or a combination thereof.

15. The printing process for making a printed absorbent article according to claim 11, wherein the ink receptive coating comprises a surfactant.

16. The printing process for making a printed absorbent article according to claim 11, wherein the printing speed is from about 1000 feet per minute to about 2200 feet per minute.

17. The printing process for making a printed absorbent article according to claim 11, wherein the amount of aqueous ink applied to the substrate through the coating is dependent on the amount of ink required to apply onto the substrate.

18. The printing process for making a printed absorbent article according to claim 11, wherein the amount of aqueous ink absorbed by the coating after applying the aqueous ink onto the substrate is the same.

19. The printing process for making a printed absorbent article according to claim 11, wherein the printed absorbent article is packaged for commercial sale.
